(19) United States
(12) Patent Application Publication
(10) Pub. No.: US 2010/0130583 A1
(43) Pub. Date: May 27, 2010

(54) PROLINAMIDE DERIVATIVES AS MODULATORS OF VOLTAGE-GATED SODIUM CHANNELS

(75) Inventors: Giuseppe Alvaro, Verona (IT); Markus Bergauer, Verona (IT); Agostino Marasco, Verona (IT)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: GLAXO GROUP LIMITED, Greenford, Middlesex (GB)

(21) Appl. No.: 12/594,265

(22) PCT Filed: Apr. 1, 2008
(86) PCT No.: PCT/EP08/53886

§ 371 Date: Oct. 1, 2009

(30) Foreign Application Priority Data
Apr. 4, 2007 (GB) 0706630.1

(51) Int. Cl. A61K 31/40; C07D 207/10; A61P 25/24; A61P 25/00; A61P 25/30

(52) U.S. Cl. 514/423; 548/537

(57) ABSTRACT

The invention relates to quaternary α-aminocarboxyamide derivatives of formula (I) and their pharmaceutically salts or solvates

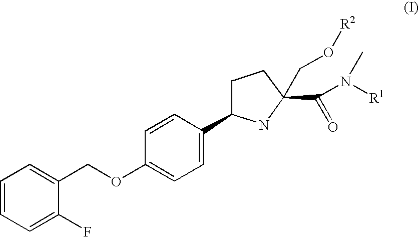

wherein
R¹ is H or CH₃; and
R² is H or CH₃,
for treating diseases and conditions mediated by modulation of voltage-gated sodium channels.

This invention relates to quaternary α-aminocarboxyamide derivatives. The invention also relates to the use of the derivatives in treating diseases and conditions mediated by modulation of voltage-gated sodium channels. In addition, the invention relates to compositions containing the derivatives and processes for their preparation.

Voltage-gated sodium channels are responsible for the initial phase of the action potential, which is a wave of electrical depolarisation usually initiated at the soma of the neuron and propagated along the nerve axon to the terminals. At the terminals, the action potential triggers the influx of calcium and the release of neurotransmitter. Drugs, such as lidocaine, that block voltage-gated sodium channels are used as local anaesthetics. Other sodium channel blockers, such as lamotrigine and carbamazepine are used to treat epilepsy. In the latter case, partial inhibition of voltage-gated sodium channels reduces neuronal excitability and reduces seizure propagation. In the case of local anaesthetics, regional block of sodium channels on sensory neurons prevents the conduction of painful stimuli. A key feature of these drugs is their use-dependent mechanism of action. The drugs are thought to stabilise an inactivated configuration of the channel that is adopted rapidly after the channel opens. This inactivated state provides a refractory period before the channel returns to its resting (closed) state ready to be reactivated. As a result, use-dependent sodium channel blockers retard the firing of neurons at high frequency, for example in response to painful stimuli, and will help to prevent repetitive firing during periods of prolonged neuronal depolarisation that might occur, for example, during a seizure. Action potentials triggered at low frequencies, for example in the heart, will not be significantly affected by these drugs, although the safety margin differs in each case, since at high enough concentrations each of these drugs is capable of blocking the resting or open states of the channels.

The voltage-gated sodium channel family is made up of 10 subtypes, four of which are brain specific, NaV1.1, 1.2, 1.3 and 1.6. Of the other subtypes, NaV1.4 is found only in skeletal muscle, NaV1.5 is specific to cardiac muscle, and NaV1.7, 1.8, and 1.9 are found predominantly in sensory neurons. The hypothesised binding site for use-dependent sodium channel blockers is highly conserved between all the subtypes. As a result, drugs such as lidocaine, lamotrigine and carbamazepine do not distinguish between the subtypes. However, selectivity can be achieved as a result of the different frequencies at which the channels normally operate.

Drugs that block voltage-gated sodium channels in a use-dependent manner are also used in the treatment of bipolar disorder, either to reduce symptoms of mania or depression, or as mood stabilisers to prevent the emergence of mood episodes. Clinical and preclinical evidence also suggests that use-dependent sodium channel blockers may help to reduce the symptoms of schizophrenia. For example, lamotrigine has been shown to reduce symptoms of psychosis induced by ketamine in healthy human volunteers, and furthermore, studies in patients suggest that the drug can augment the antipsychotic efficacy of some atypical antipsychotic drugs, such as clozapine or olanzapine. It is hypothesised that efficacy in these psychiatric disorders may result in part from a reduction of excessive glutamate release. The reduction in glutamate release is thought to be a consequence of use-dependent sodium channel inhibition in key brain areas, such as the frontal cortex. However, interaction with voltage-gated calcium channels may also contribute to the efficacy of these drugs.

International published patent application WO05/000309 (Ionix Pharmaceuticals Limited) discloses the use of compounds of formula (I), wherein R₁ is an organic substituent, X₁ and X₂ are direct bonds or spacer moieties, Ar is aryl or heteroaryl and Y is a substituted aminoalkyl group or a heteroaryl-, heterocyclyl- or phenyl-containing moiety:

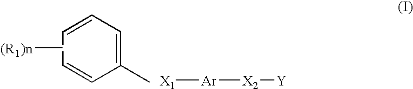

Such compounds are inhibitors of sensory neurone specific sodium channels and are said to be useful in the treatment of chronic and acute pain, tinnitus, bowel disorders, bladder dysfunction and demyelinating diseases.

International published patent application WO04/083189 (Merck & Co.) discloses biaryl substituted triazole compounds of formula (I), (II) and (III) as sodium channel blockers:

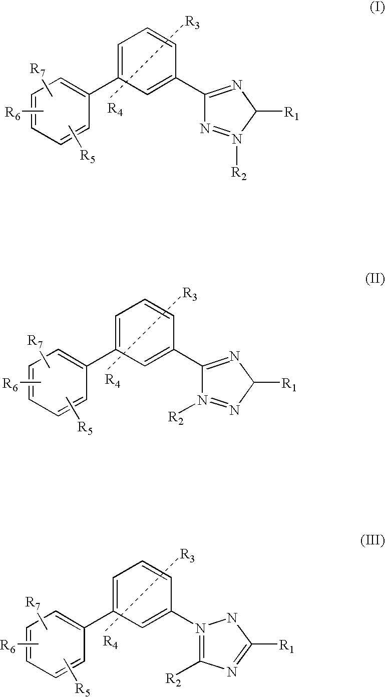

Such compounds are said to be useful in the treatment of conditions associated with sodium channel activity including, for example, acute pain, chronic pain, visceral pain, epilepsy, irritable bowel syndrome, depression and others.

International published patent application WO04/092140 (Merck & Co.) discloses biaryl substituted pyrazoles of formula (I), (II), (III) and (IV) as sodium channel blockers:

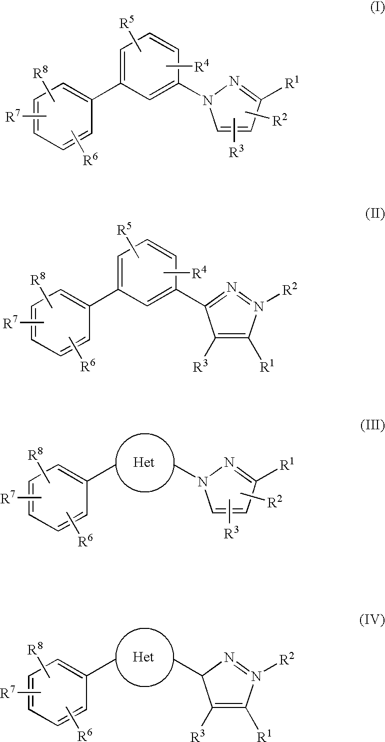

The compounds are said to be useful in the treatment of conditions including acute pain, chronic pain, visceral pain, inflammatory pain and neuropathic pain.

International published patent application WO04/094395 (Merck & Co.) discloses biaryl substituted thiazoles, oxazoles and imidazoles of formula (I) as sodium channel blockers:

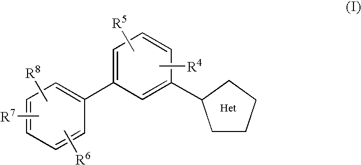

The compounds are said to be useful in the treatment of conditions including acute pain, chronic pain, visceral pain, inflammatory pain and neuropathic pain.

International patent application WO04/026826 (F. Hoffman La Roche AG) discloses 4-pyrrolidinophenyl-benzyl ether derivatives of formula (I):

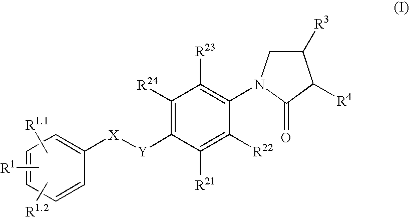

The compounds are said to be monoamine oxidase B inhibitors and are said to be useful in the treatment of conditions such as Alzheimer's disease or senile dementia.

The object of the present invention is to identify alternative compounds which modulate voltage-gated sodium channels.

In one embodiment, the compounds will be use dependent sodium channel inhibitors.

In another embodiment, the compounds will be a subtype NaV1.3 sodium channel use dependent inhibitors.

In another embodiment, the present invention provides compounds which modulate voltage-gated sodium channels but which do not exhibit Monoamine Oxidase B inhibition.

According to a first aspect, the invention provides a compound of formula (I), or a pharmaceutically acceptable salt or solvate thereof:

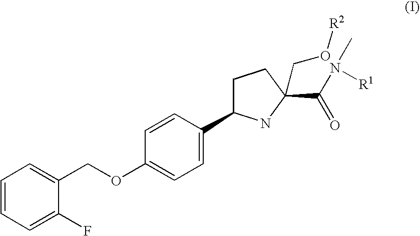

wherein

### R¹ is H or CH₃; and

### R² is H or CH₃.

In one embodiment, the compounds of formula (I) are selected from the list consisting of:
(5R)-5-{4-[(2-fluorobenzyl)oxy]phenyl}-2-(methoxymethyl)-N-methyl-L-prolinamide;
(5R)-5-{4-[(2-fluorobenzyl)oxy]phenyl}-2-(methoxymethyl)-N,N-dimethyl-L-prolinamide;
(5R)-5-{4-[(2-fluorobenzyl)oxy]phenyl}-2-(hydroxymethyl)-N,N-dimethyl-L-prolinamide; and
(5R)-5-{4-[(2-fluorobenzyl)oxy]phenyl}-2-(hydroxymethyl)-N-methyl-L-prolinamide;
   or a pharmaceutically acceptable salt or solvate thereof.

The pharmaceutically or veterinarily acceptable salts of the compounds of the invention which contain a basic centre are, for example, non-toxic acid addition salts formed with inorganic acids such as hydrochloric, hydrobromic, hydroiodic, sulfuric and phosphoric acid, with carboxylic acids or with organo-sulfonic acids. Examples include the HCl, HBr, HI, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. For reviews on suitable pharmaceutical salts see Berge et al, J. Pharm, Sci., 66, 1-19, 1977; P L Gould, International Journal of Pharmaceutics, 33 (1986), 201-217; and Bighley et al, Encyclopedia of Pharmaceutical Technology, Marcel Dekker Inc, New York 1996, Volume 13, page 453-497.

In another embodiment of the invention, the pharmaceutically acceptable salt of the compound of formula (I) is the hydrochloride salt.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Pharmaceutically acceptable solvates of the compound of the invention are within the scope of the invention.

Hereinafter, compounds and their pharmaceutically acceptable salts and solvates defined in any aspect of the invention (except intermediate compounds in chemical processes) are referred to as “compounds of the invention”.

The pharmaceutically acceptable solvates of the compounds of the invention include the hydrates thereof.

Also included within the scope of the compounds and various salts of the invention are polymorphs thereof.

The compounds of the invention may exist in one or more tautomeric forms. All tautomers and mixtures thereof are included in the scope of the present invention.

The invention also includes all suitable isotopic variations of a compound of the invention. An isotopic variation of a compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples hereafter using appropriate isotopic variations of suitable reagents.

Compounds of the invention may be prepared in a variety of ways. In the following reaction schemes and hereafter, unless otherwise stated R¹ and R² are as defined in the first aspect. These processes form further aspects of the invention.

Throughout the specification, general formulae are designated by Roman numerals (I), (II), (III), (IV) etc. Subsets of these general formulae are defined as (Ia), (Ib), (Ic) etc. . . . (IVa), (IVb), (IVc) etc.

Hydrochloride salts of compounds of formula (I) may be prepared according to Reaction Scheme 1 by reacting compounds of formula (I) with an excess (such as 2.5 equivalents) of HCl in diethylether. Typical reaction conditions comprise reacting (I) in a suitable solvent (such as EtOAc or methanol) at room temperature.

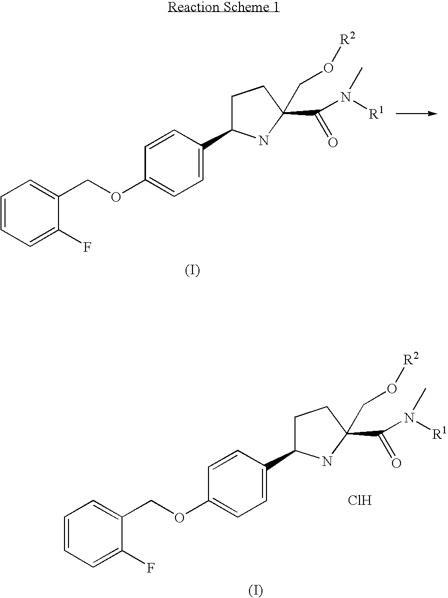

Compounds of general formula (I) may be generated according to Reaction Scheme 2 by treating compounds of general formula (II) dissolved in a polar protic solvent (such as methanol) in the presence of a catalyst (such as palladium on charcoal) in a hydrogen atmosphere (1 atm) at room temperature.

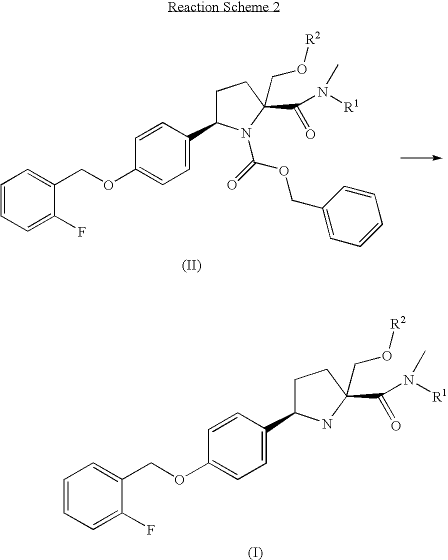

The compounds of general formula (IIa), with R²≠H, may be generated according to Reaction Scheme 3 by reacting compounds of formula (IIIa), with R²≠H, with NHMeR¹ in the presence of a base (such as diisopropylethyl amine) and a suitable reagent to activate the carboxylic acid function (such as TBTU) in an aprotic solvent (such as DMF) at temperatures ranging from 0° C. to room temperature.

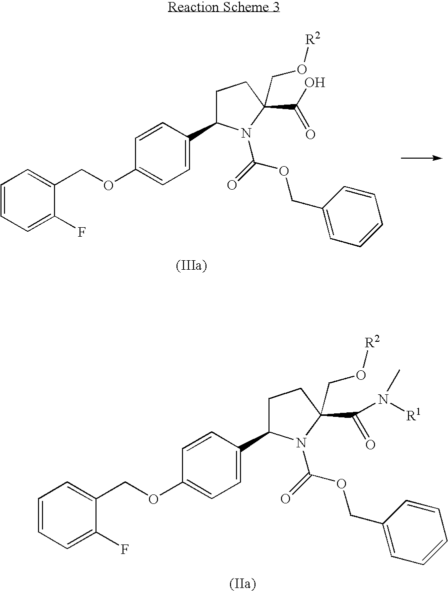

The compounds of general formula (IIIa) may be generated according to Reaction Scheme 4 by reacting compounds of general formula (IVa), R being a lower unbranched alkyl chain (such as methyl or ethyl), with a base (such as LiOH.H₂O) in a suitable solvent (such as THF) at temperatures ranging from room temperature to reflux with or without microwave heating.

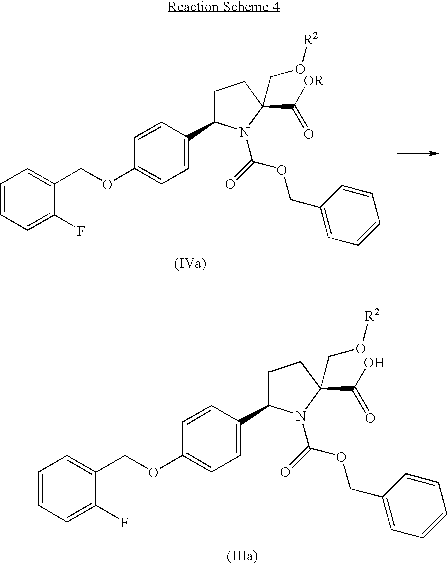

The compounds of general formula (IVa) may be generated according to Reaction Scheme 5 by reacting compounds of general formula (V), with a base (such as lithium diisopropylamide) in a suitable solvent (such as THF) at low temperatures (such as −40° C.) followed by reaction with a suitable nucleophile YCH₂OR² (such as MOMCl), Y being a leaving group (such as Cl, Br, or OSO₂CH₃) at temperatures ranging from −78° C. to room temperature.

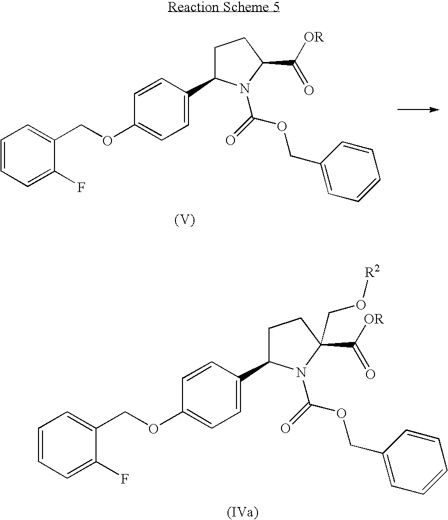

Compounds of general formula (V) may be generated according to Reaction Scheme 6 by reacting compounds of general formula (VI) with benzyl chloroformate in a chlorinated solvent (such as dichloromethane) in the presence of a suitable base (such as diisopropylethyl amine) at 0° C. to room temperature.

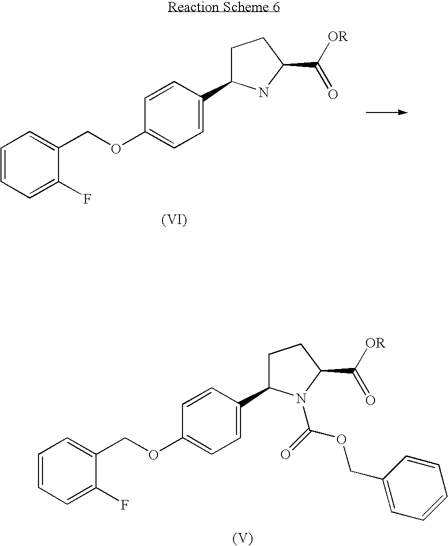

Compounds of formula (VI) can be prepared according to Reaction Scheme 7 by reacting compounds of formula (VII) with Pt/C under an atmosphere of hydrogen at elevated pressure (such as 2 atm) in a suitable solvent (such as EtOAc) at room temperature.

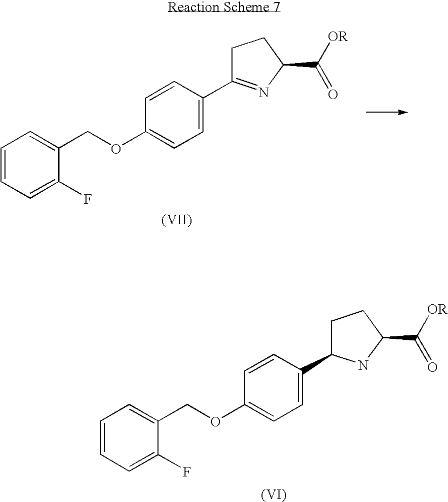

Compounds of formula (VII) may be prepared according to Reaction Scheme 8 by reacting compounds of formula (VIII) with trifluoroacetic acid in a halogenated hydrocarbon solvent (such as DCM) at a temperature ranging from 0° C. to room temperature. As an alternative, compounds of formula (VII) may be prepared from compounds of formula (IX) using a metal catalyst such as silver-(I)-triflate in N,N-dimethylformamide, tetrahydrofuran, acetonitrile or solvents having similar physicochemical properties at temperatures ranging from 0° C. to reflux. Compounds of formula (IX) are readily prepared by analogous procedures to that described in the literature (van Esseveldt et al, Journal of Organic Chemistry 2005, 70, 1791-1795 and references cited therein). The preparation for the relevant starting material is known in the literature or, alternatively, methods described below may be used.

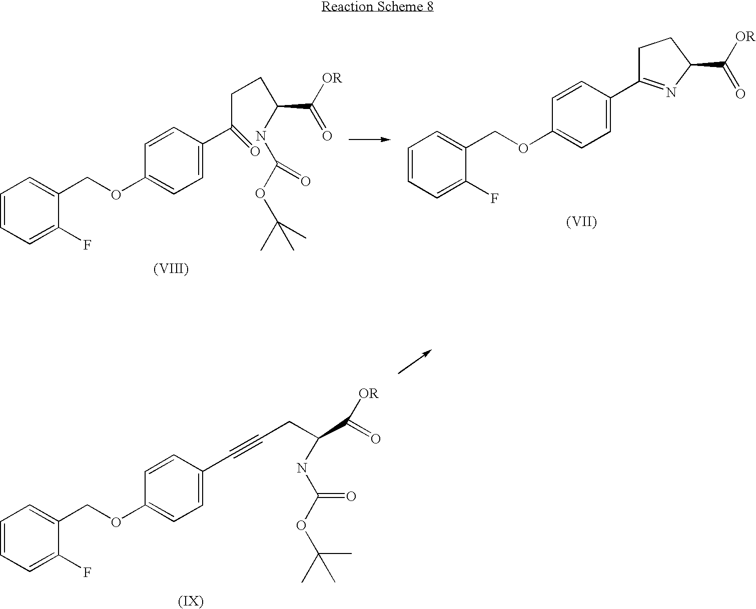

Compounds of formula (VIII) may be obtained according to Reaction Scheme 9. Typical reaction conditions comprise reacting compounds of formula (X) with a suitable metallated compound of formula (XI) where X is Cl, Br or I in a suitable aprotic solvent (such as diethyl ether or THF) at low temperature (such as −60° C.).

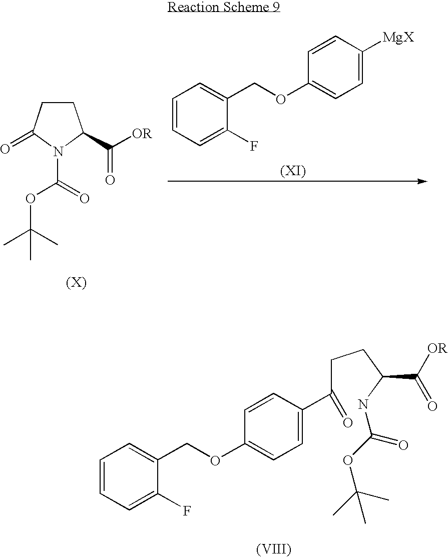

The compounds of general formula (XI) may be generated according to Reaction Scheme 10 by reacting the appropriate compound of general formula (XII) with magnesium metal in THF. Typical reaction conditions comprise reaction at a temperature ranging from room temperature to 65° C., in a suitable solvent (such as ether or THF).

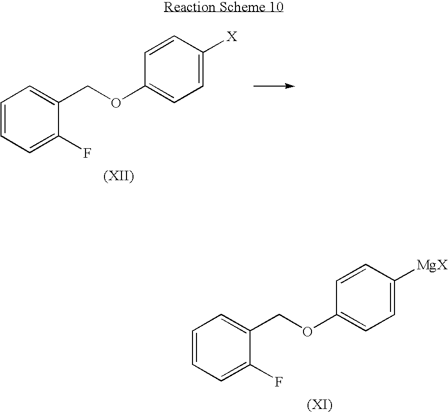

Compounds of general formula (XII) may be synthesized following the procedures described in Reaction Scheme 11 by reacting compounds of formula (XIII) with the appropriate compound of general formula (XIV), wherein Z is a suitable leaving group (such as Cl, Br, I, or OSO₂CH₃) in the presence of a suitable base (such as potassium carbonate) in a suitable solvent (such as acetone or acetonitrile) at a temperature ranging from room temperature to reflux. Compounds of general formula (XIII) and (XIV) are either commercially available or may be prepared with procedures described in the literature or methods known to the skilled person.

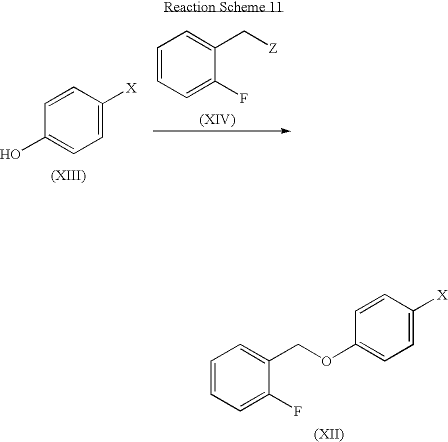

Compounds of formula (X) may be prepared according to Reaction Scheme 12 by reacting compounds of formula (XV) with a reagent suitable to transfer a BOC group onto an amine (such as di-tert-butyl dicarbonate) in the presence of a base (such as 4-DMAP) in an aprotic solvent (such as DCM) at room temperature.

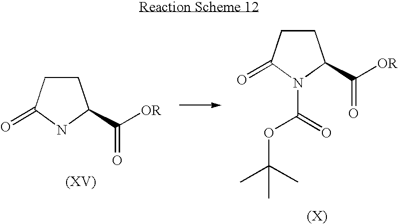

Compounds of general formula (XV) are either commercially available or can be obtained via synthetic procedures known to the skilled person.

Compounds of general formula (Ia), wherein R² is H, may be prepared according to Reaction Scheme 13 by treating compounds of general formula (XVI), wherein P is a suitable silicon protecting group (such as tert-butyldimethylsilyl), with tetrabutylammonium fluoride in a suitable solvent (such as THF) at room temperature.

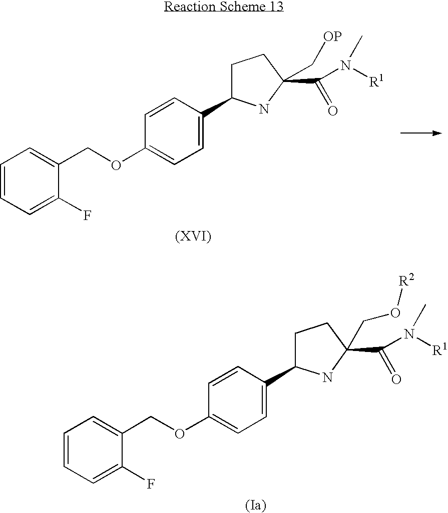

Compounds of general formula (XVI) may be generated according to Reaction Scheme 14 by treating compounds of general formula (XVII) dissolved in a polar protic solvent (such as methanol) in the presence of a catalyst (such as palladium on charcoal) in a hydrogen atmosphere (1 atm) at room temperature.

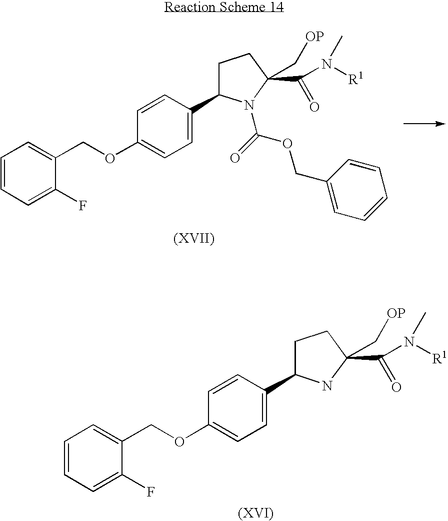

The compounds of general formula (XVII) may be generated according to Reaction Scheme 15 by reacting compounds of formula (XVIII) with NHMeR¹ in the presence of a base (such as diisopropylethyl amine) and a suitable reagent to activate the carboxylic acid function (such as TBTU) in an aprotic solvent (such as DMF) at temperatures ranging from 0° C. to room temperature.

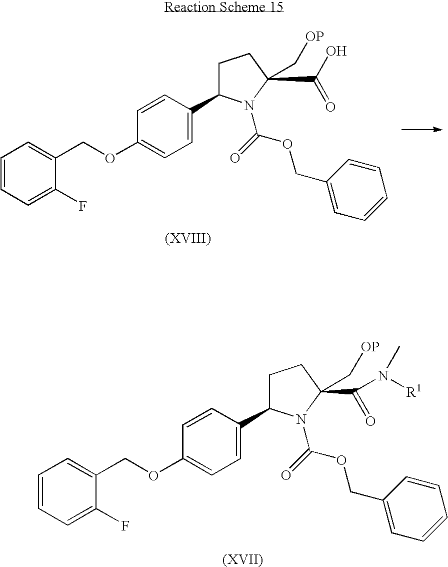

The compounds of general formula (XVIII) may be generated according to Reaction Scheme 16 by reacting compounds of general formula (XIX) with a base (such as LiOH.H₂O) in a suitable solvent (such as THF) at temperatures ranging from room temperature to reflux with or without microwave heating.

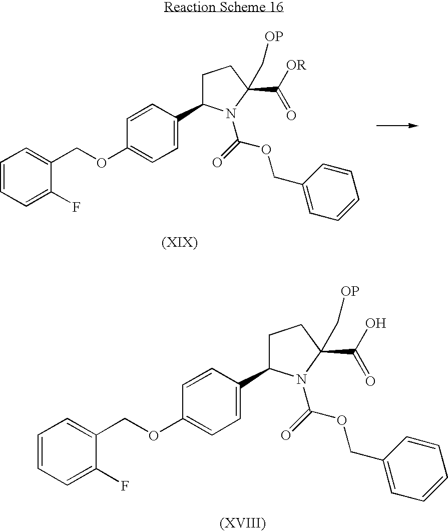

Compounds of general formula (XIX) may be generated according to Reaction Scheme 17 by reacting compounds of general formula (XX) with a suitable silyl chloride or triflate (such as tert-butyldimethyl silyl triflate) in the presence of a suitable base (such as 2,6-dimethylpyridine) in a suitable solvent (such as DCM) at 0° C. to room temperature.

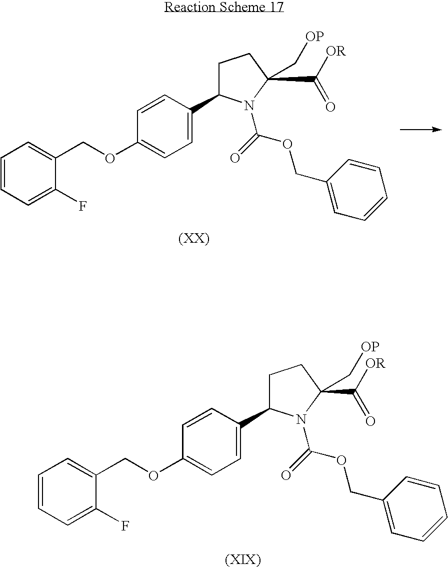

Compounds of formula (XX) may be generated according to Reaction Scheme 18 by reacting compounds (V) with a base (such as LiHMDS) in a aprotic solvent (such as THF) at low temperatures (such as −40° C.) followed by treatment with a suitable acylating agent (such as ethyl formate). The resulting compound, after a suitable workup is applied, may be immediately dissolved in an alcoholic solvent (such as methanol, ethanol or isopropylalcohol) followed by treatment with a suitable reducing agent (such as NaBH₄) at temperatures ranging from 0° C. to room temperature.

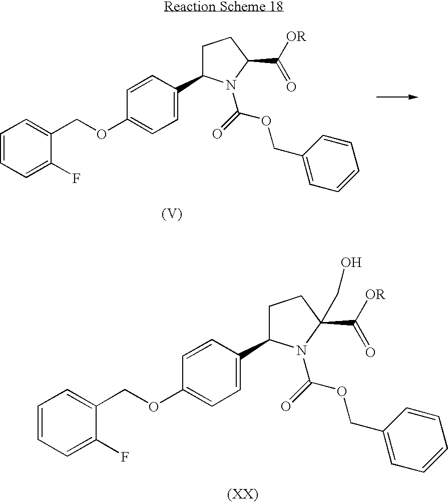

As discussed hereinabove, it is believed that compounds of the invention may be useful for the treatment of diseases and conditions mediated by modulation of voltage-gated sodium channels.

Therefore, according to a further aspect, the invention provides compounds of the invention for use as a medicament, preferably a human medicament.

According to a further aspect the invention provides the use of compounds of the invention in the manufacture of a medicament for treating or preventing a disease or condition mediated by modulation of voltage-gated sodium channels.

Without wishing to be bound by theory, diseases or conditions that may be mediated by modulation of voltage-gated sodium channels are selected from the list consisting of [the numbers in brackets after the listed diseases below refer to the classification code in Diagnostic and Statistical Manual of Mental Disorders, 4th Edition, published by the American Psychiatric Association (DSM-IV) and/or the International Classification of Diseases, 10th Edition (ICD-10)]:
i) Depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90):
ii) Schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).
iii) Anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00):
iv) Substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide:
v) Enhancement of cognition including the treatment of cognition impairment in other diseases such as schizophrenia, bipolar disorder, depression, other psychiatric disorders and psychotic conditions associated with cognitive impairment, e.g. Alzheimer's disease:
vi) Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; sleep apnea and jet-lag syndrome:
vi) Eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; Binge Eating Disorder; and Eating Disorder Not Otherwise Specified (307.50):
vii) Autism Spectrum Disorders including Autistic Disorder (299.00), Asperger's Disorder (299.80), Rett's Disorder (299.80), Childhood Disintegrative Disorder (299.10) and Pervasive Disorder Not Otherwise Specified (299.80, including Atypical Autism).
viii) Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23):
ix) Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81), Avoidant Personality Disorder (301.82), Dependent Personality Disorder (301.6), Obsessive-Compulsive Personality Disorder (301.4) and Personality Disorder Not Otherwise Specified (301.9): and
x) Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).
xi) Impulse control disorder including: Intermittent Explosive Disorder (312.34), Kleptomania (312.32), Pathological Gambling (312.31), Pyromania (312.33), Trichotillomania (312.39), Impulse-Control Disorders Not Otherwise Specified (312.3), Binge Eating, Compulsive Buying, Compulsive Sexual Behaviour and Compulsive Hoarding.

In another embodiment, diseases or conditions that may be mediated by modulation of voltage gated sodium channels are depression or mood disorders

In another embodiment, diseases or conditions that may be mediated by modulation of voltage gated sodium channels are substance related disorders.

In a further embodiment, diseases or conditions that may be mediated by modulation of voltage gated sodium channels are Bipolar Disorders (including Bipolar I Disorder, Bipolar II Disorder (i.e. Recurrent Major Depressive Episodes with Hypomanic Episodes) (296.89), Cyclothymic Disorder (301.13) or Bipolar Disorder Not Otherwise Specified (296.80)).

In a still further embodiment, diseases or conditions that may be mediated by modulation of voltage gated sodium channels are Nicotine-Related Disorders such as Nicotine Dependence (305.1), Nicotine Withdrawal (292.0) or Nicotine-Related Disorder Not Otherwise Specified (292.9).

In an embodiment, compounds of the invention may be useful as analgesics. For example they may be useful in the treatment of chronic inflammatory pain (e.g. pain associated with rheumatoid arthritis, osteoarthritis, rheumatoid spondylitis, gouty arthritis and juvenile arthritis); musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

Compounds of the invention may be useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Compounds of the invention may also be useful in the amelioration of inflammatory disorders, for example in the treatment of skin conditions (e.g. sunburn, burns, eczema, dermatitis, psoriasis); ophthalmic diseases; lung disorders (e.g. asthma, bronchitis, emphysema, allergic rhinitis, non-allergic rhinitis, cough, respiratory distress syndrome, pigeon fancier's disease, farmer's lung, chronic obstructive pulmonary disease, (COPD); gastrointestinal tract disorders (e.g. Crohn's disease, ulcerative colitis, coeliac disease, regional ileitis, irritable bowel syndrome, inflammatory bowel disease, gastroesophageal reflux disease); other conditions with an inflammatory component such as migraine, multiple sclerosis, myocardial ischemia.

Compounds of the invention may also be useful in the treatment and/or prevention of disorders treatable and/or preventable with anti-convulsive agents, such as epilepsy including post-traumatic epilepsy, obsessive compulsive disorders (OCD), sleep disorders (including circadian rhythm disorders, insomnia & narcolepsy), tics (e.g. Giles de la Tourette's syndrome), ataxias, muscular rigidity (spasticity), and temporomandibular joint dysfunction.

Compounds of the invention may also be useful in the treatment of bladder hyperrelexia following bladder inflammation.

Compounds of the invention may also be useful in the treatment of neurodegenerative diseases and neurodegeneration such as dementia, particularly degenerative dementia (including senile dementia, Alzheimer's disease, Pick's disease, Huntington's chorea, Parkinson's disease and Creutzfeldt-Jakob disease, motor neuron disease); The compounds may also be useful for the treatment of amyotrophic lateral sclerosis (ALS) and neuroinflamation.

Compounds of the invention may also be useful in neuroprotection and in the treatment of neurodegeneration following stroke, cardiac arrest, pulmonary bypass, traumatic brain injury, spinal cord injury or the like.

Compounds of the invention may also be useful in the treatment of tinnitus, and as local anaesthetics.

The compounds of the invention may also be used in combination with other therapeutic agents. The invention thus provides, in a further aspect, a combination comprising a compound of the invention or a pharmaceutically acceptable derivative thereof together with a further therapeutic agent.

When a compound of the invention or a pharmaceutically acceptable derivative thereof is used in combination with a second therapeutic agent active against the same disease state the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. The compounds of the present invention may be used in combination with other antithrombotic drugs such as thrombin inhibitors, thromboxane receptor antagonists, prostacyclin mimetics, phosphodiesterase inhibitors, fibrinogen antagonists, thrombolytic drugs such as tissue plaminogen activator and streptokinase, non-steroidal anti-inflammatory drugs such as aspirin, and the like.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations by any convenient route.

When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

The compounds of the invention may be used in combination with the following agents to treat or prevent psychotic disorders: i) antipsychotics; ii) drugs for extrapyramidal side effects, for example anticholinergics (such as benztropine, biperiden, procyclidine and trihexyphenidyl), antihistamines (such as diphenhydramine) and dopaminergics (such as amantadine); iii) antidepressants; iv) anxiolytics; and v) cognitive enhancers for example cholinesterase inhibitors (such as tacrine, donepezil, rivastigmine and galantamine).

The compounds of the invention may be used in combination with antidepressants to treat or prevent depression and mood disorders.

The compounds of the invention may be used in combination with the following agents to treat or prevent bipolar disease: i) mood stabilisers; ii) antipsychotics; and iii) antidepressants.

The compounds of the invention may be used in combination with the following agents to treat or prevent anxiety disorders: i) anxiolytics; and ii) antidepressants.

The compounds of the invention may be used in combination with the following agents to improve nicotine withdrawal and reduce nicotine craving: i) nicotine replacement therapy for example a sublingual formulation of nicotine beta-cyclodextrin and nicotine patches; and ii) bupropion.

The compounds of the invention may be used in combination with the following agents to improve alcohol withdrawal and reduce alcohol craving: i) NMDA receptor antagonists for example acamprosate; ii) GABA receptor agonists for example tetrabamate; and iii) Opioid receptor antagonists for example naltrexone.

The compounds of the invention may be used in combination with the following agents to improve opiate withdrawal and reduce opiate craving: i) opioid mu receptor agonist/opioid kappa receptor antagonist for example buprenorphine; ii) opioid receptor antagonists for example naltrexone; and iii) vasodilatory antihypertensives for example lofexidine.

The compounds of the invention may be used in combination with the following agents to treat or prevent sleeping disorders: i) benzodiazepines for example temazepam, lormetazepam, estazolam and triazolam; ii) non-benzodiazepine hypnotics for example zolpidem, zopiclone, zaleplon and indiplon; iii) barbiturates for example aprobarbital, butabarbital, pentobarbital, secobarbita and phenobarbital; iv) antidepressants; v) other sedative-hypnotics for example chloral hydrate and chlormethiazole.

The compounds of the invention may be used in combination with the following agents to treat anorexia: i) appetite stimulants for example cyproheptidine; ii) antidepressants; iii) antipsychotics; iv) zinc; and v) premenstrual agents for example pyridoxine and progesterones.

The compounds of the invention may be used in combination with the following agents to treat or prevent bulimia: i) antidepressants; ii) opioid receptor antagonists; iii) antiemetics for example ondansetron; iv) testosterone receptor antagonists for example flutamide; v) mood stabilisers; vi) zinc; and vii) premenstrual agents.

The compounds of the invention may be used in combination with the following agents to treat or prevent autism: i) antipsychotics; ii) antidepressants; iii) anxiolytics; and iv) stimulants for example methylphenidate, amphetamine formulations and pemoline.

The compounds of the invention may be used in combination with the following agents to treat or prevent ADHD: i) stimulants for example methylphenidate, amphetamine formulations and pemoline; and ii) non-stimulants for example norepinephrine reuptake inhibitors (such as atomoxetine), alpha 2 adrenoceptor agonists (such as clonidine), antidepressants, modafinil, and cholinesterase inhibitors (such as galantamine and donezepil).

The compounds of the invention may be used in combination with the following agents to treat personality disorders: i) antipsychotics; ii) antidepressants; iii) mood stabilisers; and iv) anxiolytics.

The compounds of the invention may be used in combination with the following agents to treat or prevent male sexual dysfunction: i) phosphodiesterase V inhibitors, for example vardenafil and sildenafil; ii) dopamine agonists/dopamine transport inhibitors for example apomorphine and buproprion; iii) alpha adrenoceptor antagonists for example phentolamine; iv) prostaglandin agonists for example alprostadil; v) testosterone agonists such as testosterone; vi) serotonin transport inhibitors for example serotonin reuptake inhibitors; v) noradrenaline transport inhibitors for example reboxetine and vii) 5-HT1A agonists, for example flibanserine.

The compounds of the invention may be used in combination with the same agents specified for male sexual dysfunction to treat or prevent female sexual dysfunction, and in addition an estrogen agonist such as estradiol.

Antipsychotic drugs include Typical Antipsychotics (for example chlorpromazine, thioridazine, mesoridazine, fluphenazine, perphenazine, prochlorperazine, trifluoperazine, thiothixine, haloperidol, molindone and loxapine); and Atypical Antipsychotics (for example clozapine, olanzapine, risperidone, quetiapine, aripirazole, ziprasidone and amisulpride).

Antidepressant drugs include serotonin reuptake inhibitors (such as citalopram, escitalopram, fluoxetine, paroxetine and sertraline); dual serotonin/noradrenaline reuptake inhibitors (such as venlafaxine, duloxetine and milnacipran); Noradrenaline reuptake inhibitors (such as reboxetine); tricyclic antidepressants (such as amitriptyline, clomipramine, imipramine, maprotiline, nortriptyline and trimipramine); monoamine oxidase inhibitors (such as isocarboxazide, moclobemide, phenelzine and tranylcypromine); and others (such as bupropion, mianserin, mirtazapine, nefazodone and trazodone).

Mood stabiliser drugs include lithium, sodium valproate/valproic acid/divalproex, carbamazepine, lamotrigine, gabapentin, topiramate and tiagabine.

Anxiolytics include benzodiazepines such as alprazolam and lorazepam.

It will be appreciated that references herein to “treatment” extend to prophylaxis, prevention of recurrence and suppression or amelioration of symptoms (whether mild, moderate or severe) as well as the treatment of established conditions.

The compound of the invention may be administered as the raw chemical but the active ingredient is preferably presented as a pharmaceutical formulation.

According to a further aspect, the invention provides a pharmaceutical composition comprising a compound of the invention, in association with one or more pharmaceutically acceptable carrier(s), diluents(s) and/or excipient(s). The carrier, diluent and/or excipient must be “acceptable” in the sense of being compatible with the other ingredients of the composition and not deleterious to the recipient thereof.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any route, and include those in a form adapted for oral, topical or parenteral administration to mammals including humans.

The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilising the compound and a sterile vehicle, water being preferred. The compound, depending on the vehicle and concentration used, can be either suspended or dissolved in the vehicle. In preparing solutions the compound can be dissolved in water for injection and filter-sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, for example from 10-60% by weight, of the active material, depending on the method of administration. Where the compositions comprise dosage units, each unit will for example contain from 5-1000 mg of the active ingredient. The dosage as employed for adult human treatment may range from 10 to 3000 mg per day depending on the route and frequency of administration. For oral administration a typical dose may be in the range of 50 to 1500 mg per day, for example 120 to 1000 mg per day.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of a compound of the invention given per day for a defined number of days, can be ascertained by those skilled in the art using conventional course of treatment determination tests.

All publications, including, but not limited to, patents and patent applications cited in this specification, are herein incorporated by reference as if each individual publication were specifically and individually indicated to be incorporated by reference herein as though fully set forth.

It will be appreciated that the invention includes the following further aspects. The embodiments described for the first aspect similarly apply to these further aspects. The diseases and conditions described above extend, where appropriate, to these further aspects:
i) A compound of the invention for use in treating or preventing a disease or condition mediated by modulation of voltage-gated sodium channels.
ii) A method of treatment or prevention of a disease or condition mediated by modulation of voltage-gated sodium channels in a mammal comprising administering an effective amount of a compound of the invention.
iii) Use of a compound of the invention in the manufacture of a medicament to treat or prevent a disease or condition mediated by modulation of voltage-gated sodium channels.
iv) Use of a compound of the invention to treat or prevent a disease or condition mediated by modulation of voltage-gated sodium channels.

### Experimentals

The invention is illustrated by the compounds D9, D11, D17, D20 and the Examples described below.

In the procedures that follow, after each starting material, reference to a description is typically provided. This is provided merely for assistance to the skilled chemist. The starting material may not necessarily have been prepared from the batch referred to.

The compounds described in the Examples described hereinafter have been prepared as a first step from stereochemically pure derivatives of methyl 5-oxo-L-prolinate. The stereochemistry of the compounds of the Descriptions and Examples have been assigned on the assumption that the pure configuration of 5-oxo-L-prolinate is maintained throughout any subsequent reaction conditions.

Compounds are named using ACD/Name PRO 6.02 chemical naming software (Advanced Chemistry Development Inc., Toronto, Ontario, M5H2L3, Canada).

Proton Magnetic Resonance (NMR) spectra were recorded either on Varian instruments at 300, 400, 500 or 600 MHz, or on a Bruker instrument at 300 MHz and 400 MHz. Chemical shifts are reported in ppm (δ) using the residual solvent line as internal standard. Splitting patterns are designed as s, singlet; d, doublet; t, triplet; q, quartet; m, multiplet; b, broad. The NMR spectra were recorded at a temperature ranging from 25 to 90° C. When more than one conformer was detected the chemical shifts for the most abundant one are reported.

HPLC analysis indicated by Rₜ(HPLC): x min, was performed on an Agilent 1100 series instrument using a Luna 3u C18(2) 100A (50×2.0 mm) column (mobile phase: 100% [water+0.05% TFA] to 95% [acetonitrile+0.05% TFA] in 8 min, flux=1 ml/min, detection wavelength 220 nm.

Total ion current (TIC) and DAD UV chromatographic traces together with MS and UV spectra associated with the peaks were taken also on a HPLC/MS Acquity™ system equipped with 2996 PDA detector and coupled to a Waters Micromass ZQ™ mass spectrometer operating in positive or negative electrospray ionisation mode. [LC/MS-ES (+/−): analyses performed using an Acquity™ HPLC BEH C18 column (50×21 mm, 1.7 μm particle size), column temperature 40° C. (mobile phase: A—water+0.1% HCOOH/B—MeCN+0.075% HCOOH, Flow rate: 1.0 mL/min, Gradient: t=0 min 3% B, t=0.05 min 6% B, t=0.57 min 70% B, t=1.4 min 99% B, t=1.45 min 3% B)]. The usage of this methodology is indicated by “HPLC” in the analytic characterization of the described compounds.

Mass spectra (MS) were taken on a 4 II triple quadrupole Mass Spectrometer (Micromass UK) or on a Agilent MSD 1100 Mass Spectrometer, operating in ES (+) and ES (−) ionization mode or on a Agilent LC/MSD 1100 Mass Spectrometer, operating in ES (+) and ES (−) ionization mode coupled with HPLC instrument Agilent 1100 Series [LC/MS-ES (+): analysis performed on a Supelcosil ABZ+Plus (33×4.6 mm, 3 μm) (mobile phase: 100% [water+0.1% HCO₂H] for 1 min, then from 100% [water+0.1% HCO₂H] to 5% [water+0.1% HCO₂H] and 95% [CH₃CN] in 5 min, finally under these conditions for 2 min; T=40° C.; flux=1 mL/min; LC/MS-ES (−): analysis performed on a Supelcosil ABZ+Plus (33×4.6 mm, 3 μm) (mobile phase: 100% [water+0.05% NH₃] for 1 min, then from 100% [water+0.05% NH₃ to 5% [water+0.05% NH₃] and 95% [CH₃CN] in 5 min, finally under these conditions for 2 min; T=40° C.; flux=1 mL/min]. In the mass spectra only one peak in the molecular ion cluster is reported.

For reactions involving microwave irradiation, a Personal Chemistry Emrys™ Optimizer was used.

Flash silica gel chromatography was carried out on silica gel 230-400 mesh (supplied by Merck AG Darmstadt, Germany) or over Varian Mega Be—Si pre-packed cartridges or over pre-packed Biotage silica cartridges.

SPE-SCX cartridges are ion exchange solid phase extraction columns by supplied by Varian. The eluent used with SPE-SCX cartridges is methanol followed by 2N ammonia solution in methanol.

In a number of preparation purification was performed using either Biotage manual flash chromatography (Flash+) or automatic flash chromatography (Horizon) systems. All these instruments work with Biotage Silica cartridge.

SPE-Si cartridges are silica solid phase extraction columns supplied by Varian.

The following table lists the abbreviations used:
BOC₂O bis(1,1-dimethylethyl) dicarbonate
CbzCl Benzyl chloroformate
DCM—Dichloromethane
DIPEA—Diisopropylethylamine
DMAP—4-(dimethylamino)pyridine
DMF—Dimethylformamide
LiHDMS lithium hexamethyl disilazide
MOM-Cl Methoxymethyl chloride O-(benzotriazol-1-yl)-N,N,N′N′-tetramethyluronium
TBTU—tetrafluoroborate
TBAF N,N,N,N-Tetrabutylammonium fluoride
THF—Tetrahydrofuran
TFA trifluoroacetic acid
MTBE methyl-t-butyl ether
Et₂O Diethyl ether
EtOAc Ethyl acetate

### Description 1

### 1-[(4-Bromophenoxy)methyl]-2-fluorobenzene (D1)

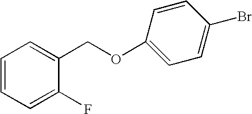

To a solution of 4-bromophenol (502.08 g) dissolved in acetone (7322 mL) was added K₂CO₃ (570 g) and then benzylbromide (523 g). The mixture was heated under reflux for 2 hrs. The reaction mixture was then cooled at 25° C., filtered and the filter cake was washed with MTBE (1046 mL). The combined filtrate was concentrated to 1000 mL and MTBE (4184 mL) were added. The mixture was washed with an aqueous 1M NaOH solution (1464 mL), then with brine (1300 mL) and was concentrated to dryness. THF (1300 mL) was added and the solvent was removed under reduced pressure to afford the title compound (902.1 g); ¹H NMR (400 MHz, DMSO-d6) δ (ppm): 7.54 (td, 1H); 7.46 (d, 2H); 7.42 (m, 1H); 7.23 (m, 2H); 7.01 (d, 2H); 5.13 (s, 2H).

### Description 2

### Methyl (2S)-2-[(tert-butoxycarbonyl)amino]-5-{4-[(2-fluorobenzyl)oxy]phenyl}-5-oxopentanoate (D2)

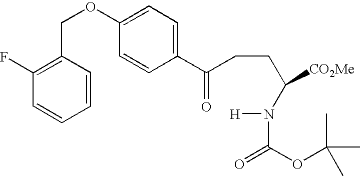

To a stirred suspension of magnesium metal (90 g) in dry THF (600 mL) under a nitrogen atmosphere at room temperature was added iodine (0.3 g). The mixture was heated to an internal temperature of 63-65° C. A solution of 1-[(4-bromophenoxy)methyl]-2-fluorobenzene (D1, 693 g) in THF (1500 mL) was added in two batches, firstly 45 mL was added in one go. Secondly, the remaining solution (1500 mL) was added dropwise. After addition, the reaction was heated at reflux for 1 h. The reaction mixture was cooled to room temperature. This reaction mixture was then added slowly to a solution of 1-tert-butyl 2-methyl (2S)-5-oxopyrrolidine-1,2-dicarboxylate (ISOCHEM, 300 g) in THF (1500 mL) cooled to −60° C., maintaining the internal temperature below −60° C. The addition was completed in 1.25 hours. The reaction mixture was stirred for a further 1 hour after addition. Isopropyl alcohol (300 mL) was then added drop-wise whilst maintaining the temperature below −60° C. A mixture of aqueous saturated ammonium chloride solution/aqueous saturated sodium chloride solution (2/1; 900 mL) was added whilst maintaining the temperature at −50° C. Water (600 mL) was added to dissolve the yellow precipitate. The organic phase was separated and was washed with aqueous 13% NaCl solution (600 mL). The organic phase was concentrated to dryness. EtOAc (1500 mL) was then added and the solution was evaporated under reduced pressure to remove water. The residue was purified by chromatography on silica gel eluting with cyclohexane/ethyl acetate (95:5 to 8:2) to afford the title compound (287 g); ¹H NMR (600 MHz, DMSO-d6) δ (ppm): 7.93 (d, 2H); 7.57 (td, 1H); 7.44 (m, 1H); 7.27 (m, 3H); 7.14 (d, 2H); 5.24 (s, 2H); 4.04 (m, 1H); 3.61 (s, 3H); 3.03 (m, 2H); 1.94 (m, 2H); 1.38 (s, 9H).

### Description 3

### Methyl (2S)-5-{4-[(2-fluorobenzyl)oxy]phenyl}-3,4-dihydro-2H-pyrrole-2-carboxylate (D3)

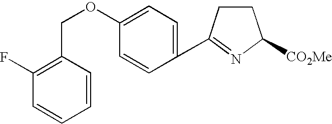

To a solution of methyl (2S)-2-[(tert-butoxycarbonyl)amino]-5-{4-[(2-fluorobenzyl)oxy]phenyl}-5-oxopentanoate (D2, 243 g) in dry DCM (2430 mL) at 0° C. was added TFA (461 mL) dropwise. The mixture was allowed to warm to room temperature and stirred for 3 hrs. Solvent and the excess TFA were removed under vacuum and the resulting dark oil was stripped with EtOAc (2×1215 mL) and left overnight under a high vacuum. The title compound (392 g) was obtained as a red oil and used in the following step without any further purification; ¹H NMR (400 MHz, DMSO-d6) δ (ppm): 8.16 (m, 2H); 7.60 (td, 1H); 7.46 (m, 1H); 7.34 (m, 2H); 7.27 (m, 2H); 5.32 (s, 2H); 5.25 (m, 1H); 3.77 (s, 3H); 3.57 (m, 2H); 2.60 (m, 1H); 2.34 (m, 1H).

### Description 4

### Methyl (5R)-5-{4-[(2-fluorobenzyl)oxy]phenyl}-L-prolinate (D4)

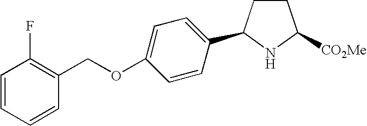

Methyl (2S)-5-{4-[(2-fluorobenzyl)oxy]phenyl}-3,4-dihydro-2H-pyrrole-2-carboxylate (D3) (392 g) was dissolved in EtOAc (3160 mL) in a hydrogenation reactor. 5% platinum on carbon (Engelhard code 44379, moisture content ca. 50%, 15.8 g) was added, the reactor filled with hydrogen gas to a pressure of 2 atm and the reaction mixture was stirred for approximately 1.5 hours. The reactor was depressurised and the spent catalyst filtered through Celite, washing through with EtOAc (2×500 mL, then further 200 mL). Aqueous saturated NaHCO₃ solution (600) was added to the filtrate, followed by aqueous 13% w/w Na₂CO₃ solution (up to pH=9, 1000 mL). The mixture was stirred for 10 minutes and phases were then allowed to separate. The aqueous phase was removed and then the organic layer was washed once with brine (600 mL). The resulting solution was concentrated to dryness and the residue was purified by flash chromatography eluting with cyclohexane/ethyl acetate (1:1) to afford the title compound (133 g); ¹H NMR (600 MHz, DMSO-d6) δ (ppm): 7.55 (dt, 1H); 7.41 (m, 1H); 7.34 (m, 2H); 7.23 (m, 2H); 6.97 (m, 2H); 5.12 (s, 2H); 4.09 (dd, 1H); 3.83 (dd, 1H); 3.66 (s, 3H); 2.97 (bs, 1H); 2.04 (m, 2H); 1.94 (m, 1H); 1.52 (m, 1H).

### Description 5

### 2-methyl 1-(phenylmethyl)(2S,5R)-5-(4-{[(2-fluorophenyl)methyl]oxy}phenyl)-1,2-pyrrolidinedicarboxylate (D5)

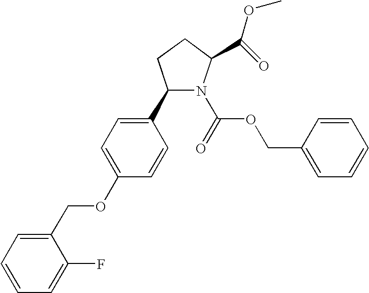

To a solution of methyl (5R)-5-{4-[(2-fluorobenzyl)oxy]phenyl}-L-prolinate (D4, 2.5 g, 7.6 mmol), prepared with an analogous method to the one described hereinabove, in DCM (dry, 25 ml) was added DIPEA (2 ml, 11.4 mmol). The solution was cooled to 0° C. and CbzCl (1.3 ml, 9.1 ml) was added dropwise. The mixture was allowed to warm to room temperature over 1 h under stirring and subsequently washed with 20% aqueous citric acid solution and brine. The organic layer was dried (Na₂SO₄), filtered and evaporated. The residue was purified by flash chromatography (EtOAc:Cyclohexanes 85:15) to afford the title compound (3.4 g). Rt (HPLC): 6.56 min; ¹H NMR (400 MHz, CDCl3) δ (ppm): 7.60-7.44 (m, 3H), 7.43-7.29 (m, 3H), 7.26-7.05 (m, 4H), 7.03-6.87 (m, 3H), 5.18-4.83 (m, 5H), 4.61-4.42 (m, 1H), 3.90-3.62 (d, 3H), 2.42-2.18 (m, 2H), 2.17-2.05 (m, 1H), 2.04-1.91 (m, 1H).

### Description 6

### 2-Methyl 1-(phenylmethyl)(2R,5R)-5-(4-{[(2-fluorophenyl)methyl]oxy}phenyl)-2-[(methyloxy)methyl]-1,2-pyrrolidinedicarboxylate (D6)

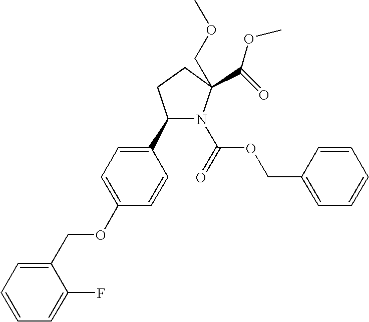

To a solution of 2-methyl 1-(phenylmethyl)(2S,5R)-5-(4-{[(2-fluorophenyl)methyl]oxy}phenyl)-1,2-pyrrolidinedicarboxylate (D5, 800 mg, 1.726 mmol), prepared with an analogous method to the one described hereinabove, in dry THF (16 ml) at −78° C. was added, dropwise, LiHMDS (1.899 ml, 1.899 mmol) 1M solution in THF and the reaction mixture was stirred for 30 min at −40° C. The mixture was cooled until −78° C. and MOM-Cl (1.311 ml, 17.26 mmol) was added. The reaction mixture was then stirred at the same temperature for 3 h.

The reaction was quenched with NaHCO₃ (sat.sol 2 ml) diluted with water and extracted with ethyl acetate (3×100 ml). The organic layer was dried (Na₂SO₄), filtered and evaporated. The residue was purified by flash chromatography (Biotage system) on silica gel using a column 25+M and cyclohexane to cyclohexane/ethyl acetate 7:3 as eluent affording the title compound (680 mg, 1.340 mmol, 78% yield) as a colourless oil. Rt (HPLC): 6.78 min; MS-ES (+): 508 [MH+], C29H30FNO6 requires 507.

### Description 7

### (5R)-5-(4-{[(2-Fluorophenyl)methyl]oxy}phenyl)-2-[(methyloxy)methyl]-1-{[(phenylmethyl)oxy]carbonyl}-L-proline (D7)

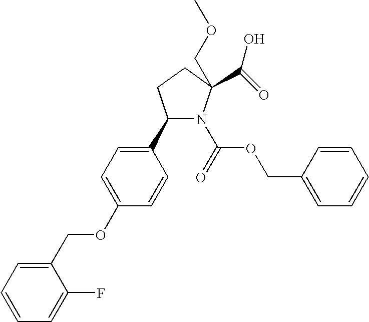

To a solution of 2-methyl 1-(phenylmethyl)(2R,5R)-5-(4-{[(2-fluorophenyl)methyl]oxy}phenyl)-2-[(methyloxy)methyl]-1,2-pyrrolidinedicarboxylate (D6, 680 mg, 1.340 mmol) in methanol (5 ml) was added a solution of lithium hydroxide (64.2 mg, 2.68 mmol) in water (2.5 ml) and the reaction mixture was stirred at 80° C. for one hour under microwave irradiation. The organic solvent was removed under vacuum and the resulting aqueous layer was adjusted to pH 5 using citrate buffer and extracted with ethyl acetate (3×80 ml). The organic layer was dried (Na₂SO₄), filtered and evaporated to afford the title compound (615 mg, 1.246 mmol, 93% yield) as a white gum. UPLC: Rt=0.88 min; MS-ES (+): 494, MS-ES (−): 492 C28H28FNO6 requires 493.

### Description 8

### Phenylmethyl (2R,5R)-5-(4-{[(2-fluorophenyl)methyl]oxy}phenyl)-2-[(methylamino)carbonyl]-2-[(methyloxy)methyl]-1-pyrrolidinecarboxylate (D8)

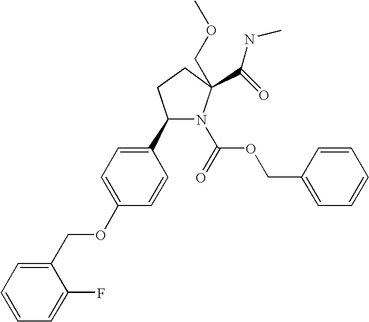

To a solution of (5R)-5-(4-{[(2-fluorophenyl)methyl]oxy}phenyl)-2-[(methyloxy)methyl]-1-{[(phenylmethyl)oxy]carbonyl}-L-proline (D7, 300 mg, 0.608 mmol) and DIPEA (0.212 ml, 1.216 mmol) in DMF (5 ml) was added TBTU (234 mg, 0.729 mmol) and the reaction mixture was stirred for 15 min at room temperature; methylamine 2M sol. in THF (0.456 ml, 0.912 mmol) was added and the mixture was stirred for 1 hour at room temperature. The reaction was quenched with brine, diluted with water and extracted with ethyl acetate (3×50 ml). The organic layer was washed with ice cold brine (3×50 ml), dried (Na₂SO₄), filtered and evaporated and the residue was purified by flash chromatography (Biotage system) on silica gel using a column 25+M and cyclohexane to cyclohexane/ethyl acetate 7:3 as eluent. The title compound (260 mg, 0.513 mmol, 84% yield) was obtained as a colourless oil. Rt (HPLC): 6.23 min, MS-ES (+): 507, C29H31FN2O5 requires 506; ¹H NMR (400 MHz, CDCl3) δ (ppm): 7.73-7.29 (m, 6H), 7.26-7.15 (m, 4H), 7.15-7.07 (m, 1H), 7.01-6.81 (m, 1H), 5.20-5.08 (m, 2H), 5.08-4.73 (m, 3H), 4.01-3.88 (m, 1H), 3.86-3.65 (m, 1H), 3.48-3.25 (m, 3H), 3.04-2.75 (m, 3H), 2.63-2.41 (m, 1H), 2.41-2.18 (m, 2H), 1.87-1.74 (m, 1H).

### Description 9

### (5R)-5-(4-{[(2-Fluorophenyl)methyl]oxy}phenyl)-N-methyl-2-[(methyloxy)methyl]-L-prolinamide (D9)

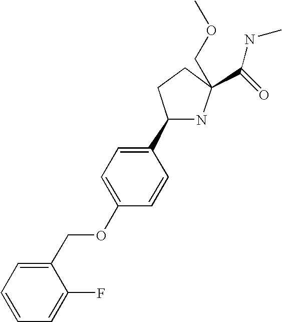

To a solution of phenylmethyl (2R,5R)-5-(4-{[(2-fluorophenyl)methyl]oxy}phenyl)-2-[(methylamino)carbonyl]-2-[(methyloxy)methyl]-1-pyrrolidinecarboxylate (D8, 260 mg, 0.513 mmol) in methanol (10 ml) was added Pd/C (30 mg, 0.282 mmol) and the reaction mixture was stirred under hydrogen (1 atm) for 20 mins. The catalyst was removed by filtration and the solvent evaporated affording the title compound (184 mg, 0.494 mmol, 96% yield) as a colourless oil. ¹H NMR (400 MHz, CDCl3) δ (ppm): 8.10-7.97 (bs, 1H), 7.57-7.50 (m, 1H), 7.38-7.29 (m, 3H), 7.22-7.15 (m, 1H), 7.15-7.07 (m, 1H), 7.02-6.95 (m, 2H), 5.19 (s, 2H), 4.33-4.25 (m, 1H), 3.98-3.92 (d, 1H), 3.40-3.33 (m, 4H), 2.87 (d, 3H), 2.79-2.66 (m, 1H), 2.24-2.16 (m, 1H), 2.11-2.02 (m, 1H), 1.86-1.75 (m, 1H), 1.70-1.63 (m, 1H).

### Description 10

### Phenylmethyl (2R,5R)-2-[(dimethylamino)carbonyl]-5-(4-{[(2-fluorophenyl)methyl]oxy}phenyl)-2-[(methyloxy)methyl]-1-pyrrolidinecarboxylate (D10)

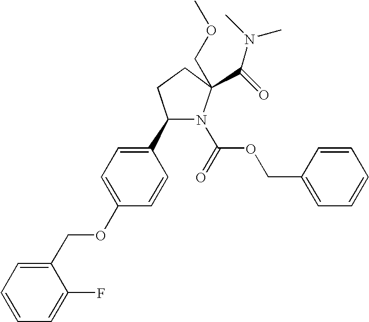

To a solution of (5R)-5-(4-{[(2-fluorophenyl)methyl]oxy}phenyl)-2-[(methyloxy)methyl]-1-{[(phenylmethyl)oxy]carbonyl}-L-proline (D7, 300 mg, 0.608 mmol) and DIPEA (0.212 ml, 1.216 mmol) in DMF (5 ml) was added TBTU (234 mg, 0.729 mmol) and the reaction mixture was stirred for 15 mins at room temperature. Then dimethylamine 2M sol. in THF (0.456 ml, 0.912 mmol) was added and the mixture was stirred for 1 hour at room temperature. The reaction was quenched with brine, diluted with water and extracted with ethyl acetate (3×50 ml). The organic layer was washed with ice cold brine (3×50 ml), dried (Na2SO4), filtered and evaporated and the residue was purified by flash chromatography (Biotage system) on silica gel using a column 25+M and cyclohexane to cyclohexane/ethyl acetate 7:3 as eluent. The title compound (270 mg, 0.519 mmol, 85% yield) was obtained as a colourless oil. UPLC: Rt=0.90 min, MS-ES (+): 521, C30H33FN2O5 requires 520.

### Description 11

### (5R)-5-(4-{[(2-Fluorophenyl)methyl]oxy}phenyl)-N,N-dimethyl-2-[(methyloxy)methyl]-L-prolinamide (D11)

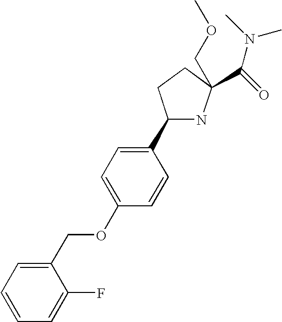

To a solution of phenylmethyl (2R,5R)-2-[(dimethylamino)carbonyl]-5-(4-{[(2-fluorophenyl)methyl]oxy}phenyl)-2-[(methyloxy)methyl]-1-pyrrolidinecarboxylate (D10, 270 mg, 0.519 mmol) in methanol (10 ml) was added Pd/C (55.2 mg, 0.519 mmol) and the reaction mixture was stirred under hydrogen (1 atm) for 20 mins. The catalyst was removed by filtration and the solvent was evaporated affording the title compound (195 mg, 0.505 mmol, 97% yield) as a colourless oil. UPLC: Rt=0.61 min, M=387, C22H27FN2O3 requires 386; ¹H NMR (400 MHz, CDCl3) δ (ppm): 7.52 (t, 1H), 7.33 (m, 3H), 7.17 (m, 1H), 7.10 (m, 1H), 6.96 (m, 2H), 5.14 (s, 2H), 4.30 (m, 1H), 3.78 (d, 2H), 3.54 (m, 1H), 3.41 (s, 3H), 3.18 (bs, 6H), 2.40 (m, 1H), 2.16 (m, 1H), 1.90 (m, 1H), 1.69 (m, 1H).

### Description 12

### 2-methyl 1-(phenylmethyl)(2R,5R)-5-(4-{[(2-fluorophenyl)methyl]oxy}phenyl)-2-(hydroxymethyl)-1,2-pyrrolidinedicarboxylate (D12)

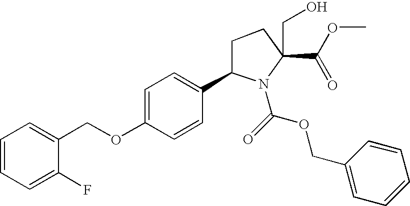

To a solution of 2-methyl 1-(phenylmethyl)(2S,5R)-5-(4-{[(2-fluorophenyl)methyl]oxy}phenyl)-1,2-pyrrolidinedicarboxylate (D5, 2.0 g, 4.31 mmol) in THF (dry, 25 ml) under argon was added at −78° C. LiHDMS (4.7 ml, 4.7 mmol) dropwise. The mixture was stirred for additional 40 min and allowed to warm to −40° C. After that the solution was brought again to −78° C., ethyl formate (8.4 ml) was added and the solution was stirred for additional 7 h at −78° C. The reaction was quenched with brine, diluted with ethyl acetate and water. The organic layer was dried and filtrated to afford a yellowish oil (ca. 2.5 g) that was used without further purification. The crude intermediate was dissolved in MeOH (20 ml) at 0° C. and sodium borohydride (160 mg, 4.3 mmol) was added. The mixture was stirred for 2 h at 0° C. The reaction was quenched with 5% NaHCO₃, the methanol evaporated and the residue diluted with water and extracted with EtOAc. The organic layer was dried and filtrated. The residue was purified via flash chromatography (Cyclohexane:EtOAc 9:1 to 7:3) to afford the title compound (1.19 g). Rt (HPLC): 6.06 min.

### Description: 13

### 2-Methyl 1-(phenylmethyl)(2R,5R)-2-({[(1,1-dimethylethyl)(dimethyl)silyl]oxy}methyl)-5-(4-{[(2-fluorophenyl)methyl]oxy}phenyl)-1,2-pyrrolidinedicarboxylate (D13)

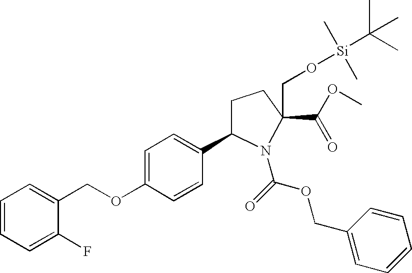

To a solution of 2-methyl 1-(phenylmethyl)(2R,5R)-5-(4-{[(2-fluorophenyl)methyl]oxy}phenyl)-2-(hydroxymethyl)-1,2-pyrrolidinedicarboxylate (D12, 600 mg, 1.216 mmol) and 2,6-lutidine (0.212 ml, 1.824 mmol) in dry DCM (5 ml) was slowly added TDBMSOTf (0.335 ml, 1.459 mmol) at 0° C. and the reaction mixture was stirred for 3 hours at room temperature. The reaction was diluted with water and extracted with DCM (3×80 ml). The organic layer was dried (Na₂SO₄), filtered and evaporated. The residue was purified by flash chromatography (Biotage system) on silica gel using a column 25+M and cyclohexane to cyclohexane/ethyl acetate 7:3 as eluent affording the title compound (730 mg, 1.201 mmol, 99% yield) as a colorless oil. UPLC: Rt=1.23 min. ¹H NMR (400 MHz, CDCl3) δ (ppm): 7.61-7.29 (m, 5H), 7.24-6.73 (m, 8H), 5.18-4.75 (m, 5H), 4.51-4.38 (m, 1H), 4.02-3.88 (d, 1H), 3.86-3.76 (s, 2H), 3.65-3.50 (s, 1H), 2.79-2.57 (m, 1H), 2.43-2.18 (m, 2H), 1.90-1.70 (m, 1H), 0.92-0.86 (m, 9H), 0.09-0.0 (m, 6H).

### Description 14

### (5R)-2-({[(1,1-Dimethylethyl)(dimethyl)silyl]oxy}methyl)-5-(4-{[(2-fluorophenyl)methyl]oxy}phenyl)-1-{[(phenylmethyl)oxy]carbonyl}-L-proline (D14)

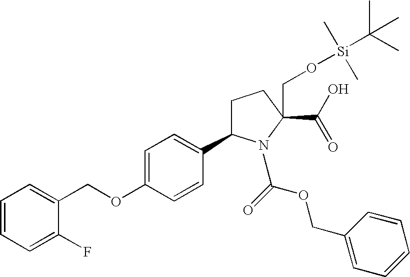

To a solution of 2-methyl 1-(phenylmethyl)(2R,5R)-2-({[(1,1-dimethylethyl)(dimethyl)silyl]oxy}methyl)-5-(4-{[(2-fluorophenyl)methyl]oxy}phenyl)-1,2-pyrrolidinedicarboxylate (D13, 730 mg, 1.201 mmol) in methanol (8 ml) was added a solution of LiOH.H₂O (101 mg, 2.402 mmol) in water (4.00 ml) and the mixture was stirred for 2 hours at 90° C. under microwave irradiation. The solvent was removed under vacuum and the residue was dissolved in water and the pH was adjusted to pH=5 with citrate buffer; this solution was extracted with ethyl acetate (2×100 ml). The organic layer was dried (Na₂SO₄), filtered and evaporated affording the title compound (590 mg, 0.994 mmol, 83% yield) as a white gum. UPLC: Rt=1.15 min.

### Description 15

### Phenylmethyl (2R,5R)-2-[(dimethylamino)carbonyl]-2-({[(1,1-dimethylethyl)(dimethyl)silyl]oxy}methyl)-5-(4-{[(2-fluorophenyl)methyl]oxy}phenyl)-1-pyrrolidinecarboxylate (D15)

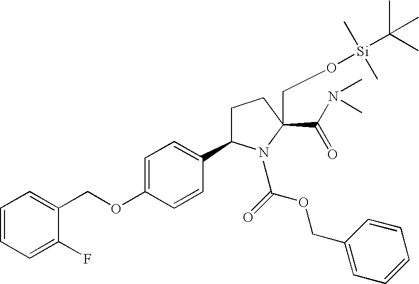

To a solution of (5R)-2-({[(1,1-dimethylethyl)(dimethyl)silyl]oxy}methyl)-5-(4-{[(2-fluorophenyl)methyl]oxy}phenyl)-1-{[(phenylmethyl)oxy]carbonyl}-L-proline (D14, 295 mg, 0.497 mmol) in DMF (4 ml) were added DIPEA (0.174 ml, 0.994 mmol) and TBTU (191 mg, 0.596 mmol) and the reaction mixture was stirred for 15 mins. at room temperature. Dimethylamine 2M Sol. in THF (0.373 ml, 0.745 mmol) was added and the reaction mixture was stirred for 1 hour at room temperature. The reaction was quenched with brine, diluted with water and extracted with ethyl acetate (3×80 ml). The organic layer was washed with ice cold brine (3×100 ml), dried (Na₂SO₄), filtered and evaporated. The residue was purified by flash chromatography (Biotage system) on silica gel using a column 25+M and cyclohexane to cyclohexane/ethyl acetate 7:3 as eluent affording the title compound (240 mg, 0.387 mmol, 78% yield) as a colourless oil. Rt (HPLC): 8.09 min; UPLC: Rt=1.18 min; ¹H NMR (400 MHz, CDCl3) δ (ppm): 7.58-7.49 (m, 1H), 7.48-7.15 (m, 8H), 7.15-7.06 (m, 1H), 7.06-6.78 (m, 3H), 5.34-5.09 (m, 3H), 5.09-4.79 (m, 2H), 4.69-4.19 (d, 1H), 4.14-4.04 (m, 1H), 3.20-2.98 (m, 3H), 2.98-2.78 (m, 3H), 2.78-2.52 (m, 1H), 2.52-2.24 (m, 2H), 2.11-1.82 (m, 1H), 0.90 (s, 9H), 0.05 (d, 6H).

### Description 16

### (5R)-2-({[(1,1-dimethylethyl)(dimethyl)silyl]oxy}methyl)-5-(4-{[(2-fluorophenyl)methyl]oxy}phenyl)-N,N-dimethyl-L-prolinamide (D16)

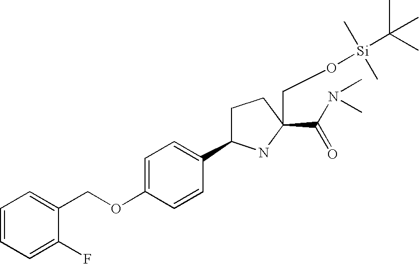

To a solution of phenylmethyl (2R,5R)-2-[(dimethylamino)carbonyl]-2-({[(1,1-dimethylethyl)(dimethyl)silyl]oxy}methyl)-5-(4-{[(2-fluorophenyl)methyl]oxy}phenyl)-1-pyrrolidinecarboxylate (D15, 240 mg, 0.387 mmol) in Methanol (10 ml) was added Pd/C 10% w/w (30 mg, 0.028 mmol) and the reaction mixture was stirred for 1 hour at r.t. under H2 atmosphere (P=1 atm). The mixture was filtered on CELITE and the solvent was evaporated to afford the title compound (185 mg, 0.380 mmol, 98% yield) as a colourless oil. UPLC: Rt=0.78 min, M=487, C27H39FN2O3Si requires 486. Rt (HPLC): 5.33 min.

### Description 17

### (5R)-5-(4-{[(2-Fluorophenyl)methyl]oxy}phenyl)-2-(hydroxymethyl)-N,N-dimethyl-L-prolinamide (D17)

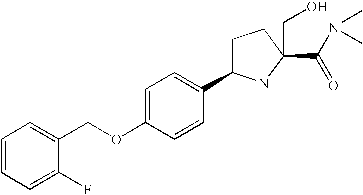

To a solution of (5R)-2-({[(1,1-dimethylethyl)(dimethyl)silyl]oxy}methyl)-5-(4-{[(2-fluorophenyl)methyl]oxy}phenyl)-N,N-dimethyl-L-prolinamide (D16, 185 mg, 0.380 mmol) in dry Tetrahydrofuran (THF) (8 ml) TBAF 1M in THF (0.418 ml, 0.418 mmol) was added dropwise at 0° C. and the reaction mixture was stirred for 1 hour at the same temperature. The reaction was quenched with a 5% NaHCO₃ solution (10 ml) and extracted with ethyl acetate. The organic layer was dried (Na₂SO₄), filtered and evaporated and the residue was purified by flash chromatography (Biotage system) on silica gel using a column 12+M and dichloromethane to DCM/MeOH 95:5 as eluent affording the title compound (70 mg, 0.188 mmol, 49.4% yield) as a white solid. UPLC: Rt=0.59 min, M=373, C21H25FN2O3 requires 372; Rt (HPLC): 3.35 min.

### Description 18

### Phenylmethyl (2R,5R)-2-({[(1,1-dimethylethyl)(dimethyl)silyl]oxy}methyl)-5-(4-{[(2-fluorophenyl)methyl]oxy}phenyl)-2-[(methylamino)carbonyl]-1-pyrrolidinecarboxylate (D18)

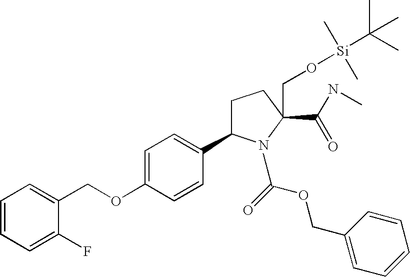

To a solution of (5R)-2-({[(1,1-dimethylethyl)(dimethyl)silyl]oxy}methyl)-5-(4-{[(2-fluorophenyl)methyl]oxy}phenyl)-1-{[(phenylmethyl)oxy]carbonyl}-L-proline (D14, 295 mg, 0.497 mmol) in N,N-Dimethylformamide (DMF) (4 ml) were added DIPEA (0.174 ml, 0.994 mmol) and TBTU (191 mg, 0.596 mmol) and the reaction mixture was stirred for 15 mins. at room temperature. Methylamine 2M sol. in THF (0.373 ml, 0.745 mmol) was added and the reaction mixture was stirred for 1 hour at room temperature. The reaction was quenched with brine, diluted with water and extracted with ethyl acetate (3×80 ml). The organic layer was washed with ice cold brine (3×100 ml), dried (Na₂SO₄), filtered and evaporated. The residue was purified by flash chromatography (Biotage system) on silica gel using a column 25+M and cyclohexane to cyclohexane/ethyl acetate 7:3 as eluent affording the title compound (215 mg, 0.354 mmol, 71.3% yield) as a colorless oil. UPLC: Rt=1.16 min; ¹H NMR (400 MHz, CDCl3) δ (ppm): 7.70-7.58 (m, 1H), 7.57-7.49 (m, 1H), 7.47-7.29 (m, 3H), 7.26-7.06 (m, 5H), 7.06-6.74 (m, 4H), 5.21-5.10 (s, 2H), 5.09-4.75 (m, 3H), 4.24-3.94 (m, 2H), 3.03-2.67 (m, 3H), 2.64-2.42 (m, 1H), 2.42-2.16 (m, 2H), 1.86-1.72 (m, 1H), 0.93 (s, 9H), 0.16-0.04 (d, 6H).

### Description 19

### (5R)-2-({[(1,1-Dimethylethyl)(dimethyl)silyl]oxy}methyl)-5-(4-{[(2-fluorophenyl)methyl]oxy}phenyl)-N-methyl-L-prolinamide (D19)

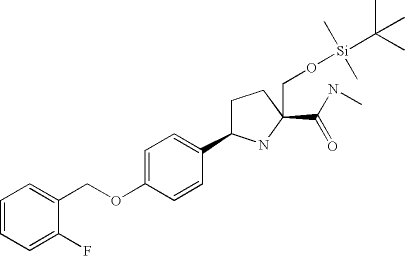

To a solution of phenylmethyl (2R,5R)-2-({[(1,1-dimethylethyl)(dimethyl)silyl]oxy}methyl)-5-(4-{[(2-fluorophenyl)methyl]oxy}phenyl)-2-[(methylamino)carbonyl]-1-pyrrolidinecarboxylate (D18, 215 mg, 0.354 mmol) in Methanol (10 ml) was added Pd/C 10% w/w (30 mg, 0.028 mmol) and the reaction mixture was stirred for 1 hour at room temperature under H2 atmosphere (P=1 atm). The mixture was filtered on CELITE and the solvent was evaporated to afford the title compound (160 mg, 0.339 mmol, 96% yield) as a colorless oil. UPLC: Rt=0.82 min, M=473, C26H37FN2O3Si requires 472; Rt (HPLC): 5.21 min.

### Description 20

### (5R)-5-(4-{[(2-Fluorophenyl)methyl]oxy}phenyl)-2-(hydroxymethyl)-N-methyl-L-prolinamide (D20)

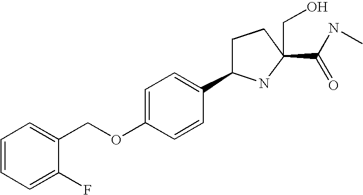

To a solution of (5R)-2-({[(1,1-dimethylethyl)(dimethyl)silyl]oxy}methyl)-5-(4-{[(2-fluorophenyl)methyl]oxy}phenyl)-N-methyl-L-prolinamide (D19, 160 mg, 0.339 mmol) in dry tetrahydrofuran (THF) (6 ml) TBAF 1M in THF (0.372 ml, 0.372 mmol) was added dropwise at 0° C. and the reaction mixture was stirred for 1 hour at the same temperature. The reaction was quenched with a 5% NaHCO₃ solution (10 ml) and extracted with ethyl acetate. The organic layer was dried (Na₂SO₄), filtered and evaporated and the residue was purified by flash chromatography (Biotage system) on silica gel using a column 12+M and dichloromethane to dichloromethane/methanol 95:5 as eluent affording the title compound (85 mg, 0.237 mmol, 70.1% yield) as a white solid. Rt (HPLC): 3.67 min, UPLC: Rt=0.57 min, M=359, C20H23FN2O3 requires 358, 1H NMR (500 MHz, DMSO-d6) δ ppm: 8.07 (m, 1H), 7.55 (t, 1H), 7.39 (m, 3H), 7.25 (m, 2H), 6.97 (m, 2H), 5.12 (s, 2H), 4.87 (t, 2H), 4.20 (m, 1H), 3.69 (q, 1H), 3.37 (q, 1H), 3.01 (d, 3H), 1.98-1.87 (m, 2H), 1.69 (m, 1H), 1.37 (m, 1H).

### EXAMPLES

### Example 1

### (5R)-5-(4-{[(2-Fluorophenyl)methyl]oxy}phenyl)-N-methyl-2-[(methyloxy)methyl]-L-prolinamide hydrochloride (E1)

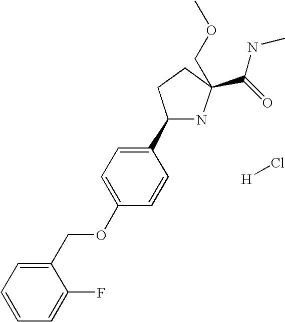

(5R)-5-(4-{[(2-Fluorophenyl)methyl]oxy}phenyl)-N-methyl-2-[(methyloxy)methyl]-L-prolinamide (D9, 184 mg) was dissolved in diethyl ether (4 ml) and HCl 1M in Et2O (0.5 ml) was added. The solvent was removed affording the title compound (200 mg, 0.489 mmol, 95% yield) as a white solid. MS-ES (+): 374, C21H25FN2O3 requires 373; 1H NMR (500 MHz, DMSO-d6) d ppm 10.04-10.26 (m, 1H) 8.64-8.85 (m, 1H) 8.16-8.33 (m, 1H) 7.57 (t, 1H) 7.46 (d, 2H) 7.41-7.48 (m, 1H) 7.23-7.30 (m, 2H) 7.11 (d, 2H) 5.18 (s, 2H) 4.57-4.76 (m, 1H) 3.98 (d, 1H) 3.76 (d, 1H) 3.30 (s, 3H) 2.75 (d, 3H) 2.29-2.38 (m, 1H) 2.19-2.31 (m, 1H) 2.03-2.17 (m, 1H) 1.80-1.96 (m, 1H).

### Example 2

### (5R)-5-(4-{[(2-Fluorophenyl)methyl]oxy}phenyl)-N,N-dimethyl-2-[(methyloxy)methyl]-L-prolinamide hydrochloride (E2)

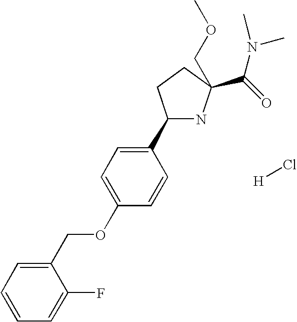

(5R)-5-(4-{[(2-fluorophenyl)methyl]oxy}phenyl)-N,N-dimethyl-2-[(methyloxy)methyl]-L-prolinamide (D11, 195 mg) was dissolved in diethyl ether (4 ml) and HCl 1M in Et2O (0.5 ml) was added. The solvent was removed affording the title compound (206 mg, 0.504 mmol, 97% yield) as a white solid. Rt (HPLC): 3.97 min, UPLC: Rt=0.58 min, M=387, C22H27FN2O3 requires 386; 1H NMR (500 MHz, DMSO-d6) δ ppm: 10.17-10.36 (m, 1H) 8.41-8.67 (m, 1H) 7.57 (t, 1H) 7.46 (d, 2H) 7.41-7.49 (m, 1H) 7.23-7.31 (m, 2H) 7.11 (d, 2H) 5.18 (s, 2H) 4.65-4.78 (m, 1H) 4.05 (d, 1H) 3.86 (d, 1H) 3.34 (s, 3H) 3.14 (s, 3H) 2.99 (s, 3H) 2.42-2.53 (m, 1H) 2.29-2.39 (m, 1H) 2.20-2.31 (m, 1H) 1.93-2.04 (m, 1H).

### Example 3

### (5R)-5-(4-{[(2-fluorophenyl)methyl]oxy}phenyl)-2-(hydroxymethyl)-N,N-dimethyl-L-prolinamide hydrochloride (E3)

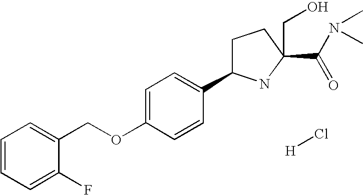

(5R)-5-(4-{[(2-fluorophenyl)methyl]oxy}phenyl)-2-(hydroxymethyl)-N, N-dimethyl-L-prolinamide (D17, 70 mg, 0.188 mmol). This solid was dissolved in diethyl ether (3 ml) and HCl 1M in Et2O (0.26 ml) was added; the suspension was triturated and the solvent removed under vacuum to afford the title compound (5R)-5-(4-{[(2-fluorophenyl)methyl]oxy}phenyl)-2-(hydroxymethyl)-N,N-dimethyl-L-prolinamide.HCl (75 mg, 0.183 mmol, 48.3% yield) as a white solid. UPLC: Rt=0.58 min, M=373, C21H25FN2O3 requires 372; 1H NMR (500 MHz, DMSO-d6) δ ppm 10.02-10.25 (m, 1H) 8.37-8.58 (m, 1H) 7.55 (t, 1H) 7.46 (d, 2H) 7.39-7.47 (m, 1H) 7.21-7.29 (m, 2H) 7.09 (d, 2H) 5.82 (t, 1H) 5.15 (s, 2H) 4.64-4.76 (m, 1H) 4.05 (dd, 1H) 3.94 (dd, 1H) 3.12 (s, 3H) 2.95 (s, 3H) 2.37-2.46 (m, 1H) 2.21-2.37 (m, 2H) 1.96-2.07 (m, 1H).

### Example 4

### (5R)-5-(4-{[(2-Fluorophenyl)methyl]oxy}phenyl)-2-(hydroxymethyl)-N-methyl-L-prolinamide hydrochloride

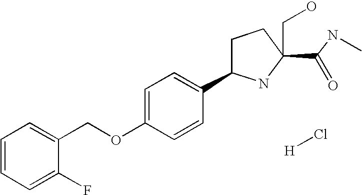

(5R)-5-(4-{[(2-fluorophenyl)methyl]oxy}phenyl)-2-(hydroxymethyl)-N-methyl-L-prolinamide (D20, 85 mg, 0.237 mmol) was dissolved in diethyl ether (3 ml) and HCl 1M in Et₂O (0.26 ml) was added; the suspension was triturated and the solvent removed under vacuum to afford the title compound (5R)-5-(4-{[(2-fluorophenyl)methyl]oxy}phenyl)-2-(hydroxymethyl)-N-methyl-L-prolinamide.HCl (90 mg, 0.228 mmol, 67.3% yield) as a white solid. Rt (HPLC): 3.68 min, UPLC: Rt=0.55 min, M=359, C20H23FN2O3 requires 358, 1H NMR (500 MHz, DMSO-d6) δ ppm: 9.97-10.21 (m, 1H) 8.58-8.82 (m, 1H) 8.05-8.25 (m, 1H) 7.57 (t, 1H) 7.46 (d, 2H) 7.41-7.50 (m, 1H) 7.22-7.32 (m, 2H) 7.12 (d, 2H) 5.69 (t, 1H) 5.19 (s, 2H) 4.57-4.79 (m, 1H) 3.76-4.06 (m, 2H) 2.75 (d, 3H) 2.23-2.35 (m, 2H) 2.06-2.21 (m, 1H) 1.83-2.00 (m, 1H).

### Biological Assay

### Na Channel Assay Protocol

The ability of the compounds of the invention to modulate the voltage-gated sodium channel subtype NaV 1.3 may be determined by the following assay.

### Cell Biology

Stable cell lines expressing hNaV1.3 channels were created by transfecting CHO cells with the pCIN5-hNav1.3 vector using the lipofectamine (Invitrogen) transfection method. pCIN5 is a bicistronic vector for the creation of mammalian cell lines that predisposes all neomycin resistant cells to express recombinant protein (see Rees S., Coote J., Stable J., Goodson S., Harris S. & Lee M. G. (1996) Biotechniques, 20, 102-112) by virtue of the recombinant cDNA being linked to the neomycin-selectable marker cDNA downstream of the CMV promoter (for full details see Chen Y H, Dale T J, Romanos M A, Whitaker W R, Xie X M, Clare J J. Cloning, distribution and functional analysis of the type III sodium channel from human brain Eur J Neurosci, 2000 December; 12, 4281-9). Cells were cultured in Iscove's modified Dulbecco's medium (Invitrogen) containing, 10% fetal bovine serum, 1% L-glutamine, 1% Penicillin-Streptomycin (Invitrogen), 1% non-essential amino acids, 2% H-T supplement and 1% G418 (Invitrogen) and maintained at 37° C. in a humidified environment containing 5% CO2 in air. Cells were liberated from the T175 culture flask for passage and harvesting using Versene (Invitrogen).

### Cell Preparation

Cells were grown to 60-95% confluence in a T75 flask. Cells were lifted by removing the growth media and incubating with 1.5 ml of warmed (37° C.) Versene (Invitrogen, 15040-066) for 6 min. Lifted cells were suspended in 10 ml of PBS (Invitrogen, 14040-133). Cell suspension was then placed into a 10-ml centrifuge tube and centrifuged for 2 min at 700 rpm. After centrifugation, the supernatant was removed and the cell pellet was resuspended in 3 ml of PBS.

### Electrophysiology

Currents were recorded at room temperature (21-23° C.) using the IonWorksHT planar array electrophysiology technology (Molecular Devices Corp.). Stimulation protocols and data acquisition were carried out using a microcomputer (Dell Pentium 4). In order to determine planar electrode hole resistances (Rp), a 10 mV, 160 ms potential difference was applied across each hole. These measurements were performed before cell addition. After cell addition a seal test was performed prior to antibiotic (amphotericin) circulation to achieve intracellular access. Leak subtraction was conducted in all experiments by applying a 160 ms hyperpolarizing (10 mV) prepulse 200 ms before the test pulses to measure leak conductance. Test pulses stepping from the holding potential of −90 mV to 0 mV were applied for 20 ms and repeated 10 times at a frequency of 10 Hz. In all experiments, the test pulse protocol was performed in the absence (pre-read) and presence (post-read) of a compound. Pre- and post-reads were separated by a compound addition followed by a 3-3.5 min incubation.

### Solutions and Drugs

The intracellular solution contained the following (in mM): K-gluconate 100, KCl 40 mM, MgCl2 3.2, EGTA 3, HEPES 5, adjusted to pH 7.25. Amphotericin was prepared as 30 mg/ml stock solution and diluted to a final working concentration of 0.1 mg/ml in internal buffer solution. The external solution was Dulbecco's PBS (Invitrogen) and contained the following (in mM): CaCl2 0.90, KCl 2.67, K3PO4 1.47, MgCl2 0.50, NaCl 138, Na3PO4 8.10, with a pH of 7.4. Compounds were prepared in DMSO as 10 mM stock solutions and subsequent 1:3 serial dilutions performed. Finally the compounds were diluted 1:100 in external solution resulting in a final DMSO concentration of 1%.

### Data Analysis

The recordings were analysed and filtered using both seal resistance (>40 MΩ) and peak current amplitude (>200pA) in the absence of compound to eliminate unsuitable cells from further analysis. Paired comparisons between pre-drug and post-drug additions were used to determine the inhibitory effect of each compound. The concentrations of compounds required to inhibit current elicited by the 1^{st} depolarising pulse by 50% (tonic pIC50) were determined by fitting of the Hill equation to the concentration response data. In addition the use-dependent inhibitory properties of the compounds were determined by assessing the effect of compounds on the 10^{th} versus 1^{st} depolarising pulse. The ratio of the 10^{th} over 1^{st} pulse was calculated in the absence and presence of drug and the % use-dependent inhibition calculated. The data was fitted using the same equation as for the tonic pIC₅₀ and the concentration producing 15% inhibition (use-dependent pUD₁₅) calculated.

The compounds of Examples 1 to 4 were tested in the above assay and gave pUD₁₅ values of 5.0 or greater.

### Monoamine Oxidase-B Assay Protocol

The protocol describes the assay for testing MAO-B inhibition. It is a fluorescence-based end-point assay using benzylamine as substrate. Oxidation of the substrate by MAO-B leads to hydrogen peroxide release, and this product is then utilised by peroxidase to convert non-fluorescent Amplex Red™ into fluorescent resorufin. The global reaction is:

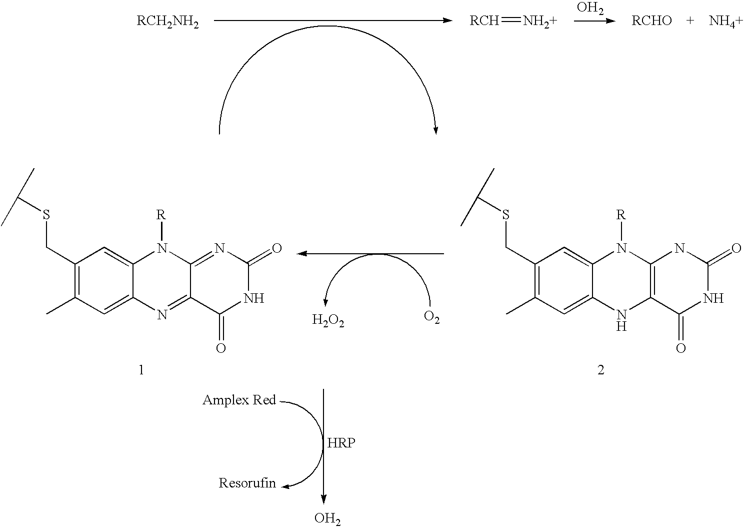

Thus inhibition of the enzyme by a test compounds leads to reduced fluorescence.

The assay uses human recombinant monoamine oxidase B that is present in microsomes from baculovirus infected insect cells (Supplied by Gentest-BD Sciences). Compounds are tested over a range of concentrations in order to determine the concentration that causes half-maximal inhibition of the enzyme activity in the assay (IC50). Pargyline (Sigma) is used as a positive control in the assay, giving an IC50 in the range 0.4-2 μM.

Dispense 0.1 μl of test compound in neat DMSO in black low volume Greiner 384-well plate. Add 5 μl of substrate solution (100 μM benzylamine (Sigma), 50 μM Amplex Red (Molecular Probes), 50 mM potassium phosphate, pH 7.4). Add 5 μl of the assay buffer (50 mM potassium phosphate, pH 7.4, 1 IU/ml horseradish peroxidase type XII (Sigma)) to blank wells. Add 5 μl of the enzyme solution (0.23 IU/ml human recombinant monoamine oxidase B, 1 IU/ml horseradish peroxidase type XII (Sigma), 50 mM potassium phosphate, pH 7.4) to remaining wells. Shake to ensure proper mixing. Incubate for 60 minutes at room temperature in darkness. Read fluorescence using Viewlux Reader (PerkinElmer; Resorufin: EX:525/20; EM:598/25; Dichroic: Bodipy)

The effect of a given compound is calculated as: % Inh=100×[(data−control1)/(control2−control1)], where control1 corresponds to the enzyme showing its maximum activity (i.e., not inhibited) and control2 corresponds to minus enzyme fluorescence in presence of HRP.

Each of Examples 1 to 4 were tested in this assay and demonstrated a pIC50 of less than 6.0.

1. A compound of formula (I) 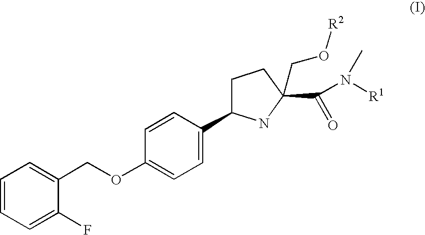
wherein
R¹ is H or CH₃; and
R² is H or CH₃;
or a pharmaceutically acceptable salt thereof.

2. A compound of formula (I) which is:
((5R)-5-{4-[(2-fluorobenzyl)oxy]phenyl}-2-(methoxymethyl)-N-methyl-L-prolinamide;
(5R)-5-{4-[(2-fluorobenzyl)oxy]phenyl}-2-(methoxymethyl)-N,N-dimethyl-L-prolinamide;
(5R)-5-{4-[(2-fluorobenzyl)oxy]phenyl}-2-(hydroxymethyl)-N,N-dimethyl-L-prolinamide; or
(5R)-5-{4-[(2-fluorobenzyl)oxy]phenyl}-2-(hydroxymethyl)-N-methyl-L-prolinamide;
or a pharmaceutically acceptable salt thereof.

3. A pharmaceutical composition comprising a compound of formula (I) as claimed in claim 1, or a pharmaceutically acceptable salt thereof, and a pharmaceutically acceptable carrier therefor.

4-12. (canceled)

13. A method of treating a disease or condition mediated by modulation of voltage-gated sodium in a mammal comprising administering an effective amount of a compound of formula (I) or a pharmaceutically acceptable salt thereof as claimed in claim 1, wherein said disorder or condition is selected from depression, a mood disorder, and a substance-related disorder and wherein said treating consists of at least one selected from the following:
(a) treating an established condition; and
(b) suppressing or ameliorating symptoms.

14. A method according to claim 13 wherein the disease or condition is depression or a mood disorder.

15. A method according to claim 14 wherein the disease or condition is a bipolar disorder.

16. A method according to claim 13 wherein the disease or condition is a substance related disorder.

17. A process to prepare a compound of formula (I), according to claim 1 comprising the reaction of a compound of general formula (II) with hydrogen in the presence of a catalyst [chemical structure]
